(19) United States
(12) Patent Application Publication
(10) Pub. No.: US 2024/0416324 A1
(43) Pub. Date: Dec. 19, 2024

(54) CESIUM-CONTAINING SUPPORTED PALLADIUM CATALYST AND ITS USE IN SELECTIVE HYDROGENATION

(71) Applicant: CLARIANT INTERNATIONAL LTD, Muttenz (CH)

(72) Inventors: Shuo Cao, Louisville, KY (US); Mingyong Sun, Louisville, KY (US)

(21) Appl. No.: 18/733,915

(22) Filed: Jun. 5, 2024

Related U.S. Application Data
(60) Provisional application No. 63/472,615, filed on Jun. 13, 2023.

(51) Int. Cl. B01J 23/63; B01J 21/04; B01J 35/61; B01J 37/02; B01J 37/08; C07C 5/08

(52) U.S. Cl. CPC B01J 23/63; B01J 21/04; B01J 35/612; B01J 37/0205; B01J 37/08; C07C 5/08; B01J 2235/30; C07C 2523/63

(57) ABSTRACT

A hydrogenation catalysts and methods of using them in hydrogenation is disclosed. More particularly, the present invention relates to hydrogenation catalysts useful for selectively hydrogenating acetylene and methylacetylene, especially in front-end streams, and methods of making and using them.

This application claims priority to U.S. 63/472,615 filed Jun. 13, 2023 the entire contents of which are hereby incorporated herein by reference.

### BACKGROUND OF THE DISCLOSURE

### 1. Field

This disclosure relates generally to hydrogenation catalysts and methods of using them in hydrogenation. More particularly, the present disclosure relates to hydrogenation catalysts useful for selectively hydrogenating acetylene and methylacetylene, especially in front-end streams, and methods of making and using them.

### 2. Technical Background

Olefins are important monomers for the production of plastics. For example, ethylene and propylene are polymerized to form polyethylene and polypropylene, respectively. Olefins such as ethylene and propylene are typically derived from petroleum products through thermal or catalytic cracking of hydrocarbons. However, cracking provides a crude olefin mixture that can contain acetylene and methylacetylene, which can interfere with the downstream polymerization of ethylene and propylene. Typically, for the polymerization of ethylene the acetylene concentration must, for example, be reduced to a value of below 1 ppm. Accordingly, it can be desirable to “clean up” this process gas to selectively convert alkynes to alkenes without substantial reduction of any olefins present or the alkynes themselves to alkanes.

There are two main reactor configurations for selective hydrogenation of acetylene in ethylene-rich streams—so-called tail-end (or back-end) processes, and front-end processes which are primarily differentiated by their positions relative to the cold box in the process layout. In the front-end configuration, the acetylene hydrogenation reactor is located before the cold box; in the tail-end it is after the cold box. In the tail-end configuration, the selective hydrogenation reactor feed typically consists mainly of C2 hydrocarbons, and stoichiometric amounts of hydrogen with respect to acetylene are added to this feed gas stream to ensure an optimal concentration of hydrogen (typically 1%-4% mol %) in the feed stream to the reactor. For front-end hydrogenation, designs can include a de-ethanizer, a de-propanizer, or raw gas, depending on the location of the reactors in the flow scheme. In the de-ethanizer design, the acetylene hydrogenation converter is typically located downstream of the de-ethanizer column and thus contains the entire C2 fraction and lighter components. In the de-propanizer design, the acetylene hydrogenation converter is typically located downstream from the de-propanizer, so the feed contains all C3 fraction and lighter components, including methyl acetylene (MA) and propadiene (PD). In the raw gas configuration, the cracked stream enters the hydrogenation reactor after acid gas removal and drying treatment but without any fractionation, and therefore the raw gas feed contains more heavy components, such as C4 and C5 hydrocarbons, including 1,3-butadiene (BD).

Regardless of design, feed to front-end acetylene selective hydrogenation typically contains 0.3-0.8% acetylene, and the converter effluent specification is normally less than 1.0 ppm. The recent trend is to operate the acetylene outlet to less than 0.3 ppm. MAPD and BD in the feeds of de-propanizer and raw gas configurations undergo the hydrogenation reaction as well. They are normally not completely converted in the acetylene hydrogenation units but will be further processed in downstream dedicated converters. As a result of relatively clean feed in the de-ethanizer configuration (without MAPD and BD), the treatment for de-ethanizer feed typically does not require as high activity as to treat de-propanizer feed, which can be often be achieved with the same catalyst at modified operating conditions.

Stringent requirements are placed on the hydrogenation process and the catalyst. On the one hand, acetylene must be removed as completely as possible by transformation into ethylene, while the hydrogenation of ethylene into ethane must be prevented. In order to ensure this result, the hydrogenation is carried out within a temperature range that is delimited on the low end by the so-called “clean-up” temperature and on the high end the so-called “run-away” temperature. The “clean-up” temperature (T1) is the operation temperature at which the hydrogenation product stream contains a desirably low level of alkyne; for the purposes of this disclosure, the desirably low level is less than 25 ppm. The “runaway temperature” (T2) is the temperature at which the hydrogenation of alkene to alkane totals 2% of the alkene in the feed stream is described as the “runaway temperature.” The operation window (OW) is typically described as the difference between the runaway temperature (T2) and the clean-up temperature (T1). The selectivity and ease of operability of a catalyst for the selective hydrogenation of acetylene depends on this operation window. As such, the activity of the hydrogenation catalyst under the process conditions must be carefully limited to avoid thermal runaway (an uncontrolled feedback loop, in which heat from the exothermic hydrogenation reaction increases the catalyst temperature, in turn increasing the rate of the hydrogenation reaction, which provides even more heat, etc.), which can result in an undesirable over reduction of ethylene to ethane and even shutdown the process due to an uncontrollable temperature rise in the reactor.

Ideally, catalysts for selective hydrogenation of acetylene would allow for a relatively large operation window (for example, 16° C.). Conventionally, front-end selective hydrogenation of acetylene present in an olefin rich mixture is performed by using optionally-promoted palladium-shell catalysts. Palladium shell catalysts, often using silver as a promoter, are primarily used as commercial catalysts for the selective hydrogenation of acetylene into ethylene in hydrocarbon streams. The palladium and the silver are supported on an inert, temperature-resistant substrate. Typically hydrogenation catalysts of acetylene include alumina-supported palladium catalysts and alumina supported palladium/silver catalysts. The addition of silver to the palladium catalyst improves the selection toward hydrogenation of acetylene, and also opens the operation window of the catalyst. However, such catalysts remain susceptible to run-away. Accordingly, there remains a need in the art for selective hydrogenation catalysts that can improve catalyst selectivity and the operation window for hydrogenation of acetylene in the presence of ethylene.

### SUMMARY

The present inventors have discovered that the catalysts described herein have especially advantageous properties for the selective hydrogenation of acetylene.

Accordingly, in one aspect, the present disclosure provides a catalyst composition comprising:
a porous support, present in an amount of at least 98 wt %; and, disposed on the support:
palladium, present in an amount of at least 0.005 wt %, e.g., in the range of 0.005 wt % to 0.2 wt %, based on the weight of the catalyst composition; and
cesium, present in an amount of at least 0.01 wt %, e.g., in the range of 0.01 wt % to 2 wt % based on the weight of the catalyst composition.
   In various desirable embodiments, the amount of cesium is at least as much as (e.g., at least twice as much as) the amount of palladium (i.e., on a weight basis).

In another aspect, the present disclosure provides a method for making a catalyst composition as described herein, the method comprising:
providing a porous support;
contacting the porous support with a first solution comprising a palladium compound and optionally a metallic compound;
drying and calcining the porous support to provide a palladium-impregnated support;
contacting the palladium impregnated support with a second solution comprising a cesium compound; and
drying and calcining the palladium-impregnated support to provide the catalyst composition.

In another aspect, the present disclosure provides a method for selective hydrogenation of alkyne (e.g., acetylene and/or methylacetylene) in an olefin feed stream comprising contacting the alkyne and hydrogen with a catalyst composition as described herein.

Other aspects of the disclosure will be apparent to the person of ordinary skill in the art based on the present the disclosure.

### BRIEF DESCRIPTION OF FIGURES

FIG. 1 is a microscope image of a cross-section of a pellet of a catalyst composition as described herein after soaking in hydrazine solution.

FIG. 2A is a scanning electron microscopy with energy dispersive x-ray analysis (SEM-EDX) image mapping aluminum of the catalyst as described herein. FIG. 2B is a SEM-EDX image mapping cesium of the catalyst as described herein.

FIG. 3A is a scanning electron microscopy with energy dispersive x-ray analysis (SEM-EDX) image of the catalyst as described herein. FIG. 3B is a SEM-EDX image mapping cesium of the catalyst as described herein.

### DETAILED DESCRIPTION

As discussed above, the selective hydrogenation of acetylene to ethylene is strictly controlled to provide effective catalysis without thermal runaway due to conversion of ethylene to ethane. The so-called operation window, i.e., a range of temperatures suitable for hydrogenation of acetylene to provide clean-up of acetylene without runaway, is strongly influenced by the hydrogenation catalyst employed in the process. Ideally, for front end hydrogenation of acetylene, it is desirable to operate the reaction with a catalyst that will provide a wide operating window (e.g., at least about 16° C.). The present inventors have found that the inclusion of cesium in a supported, optionally-promoted palladium catalyst significantly increases the operating window for selective hydrogenation. Additionally, the present inventors have found that no wet reducing agent is required when making the catalyst of the disclosure as otherwise described herein.

As described above, the present disclosure provides a catalyst composition including a porous support, palladium, cesium, and optionally, a metallic promoter. In one aspect, the disclosure provides a catalyst composition that includes
a porous support, present in an amount of at least 98 wt %; and, disposed on the support:
palladium, present in an amount of at least 0.005 wt %, e.g., in the range of 0.005 wt % to 0.2 wt %, based on the weight of the catalyst composition; and
cesium, present in an amount of at least 0.01 wt %, e.g., in the range of 0.01 wt % to 2 wt % based on the weight of the catalyst composition.
   In various desirable embodiments, the amount of cesium is at least as much as the amount of palladium (e.g., on a weight basis).

A variety of supports can be used. In various embodiments, the porous support is a support formed of alumina, silica, titania, or any mixture thereof, e.g., comprising at least 90 wt %, at least 95 wt % or at least 99 wt % of one or more of alumina, silica and titania. As used herein, terms related to oxides, including, e.g., “mixed oxide,” “alumina,” “silica,” “titania,” etc., includes stable oxides in all forms and crystalline phases, even if not perfectly stoichiometric. Unless otherwise indicated, regardless of the actual stoichiometry of the oxide, oxides are calculated as the most stable oxide for purposes of weight percent determinations. For example, the person of ordinary skill in the art will appreciate that a non-stoichiometric oxide of aluminum, or even another form of aluminum, may still be calculated as Al₂O₃. Moreover, unless otherwise indicated, the compositions are described on an as-calcined basis.

While a variety of supports can be used, in various desirable embodiments, the support is an alumina support, e.g., comprising at least 90 wt % alumina, at least 95 wt % alumina, or at least 99 wt % alumina. In various especially desirable embodiments, the support is an alpha-alumina support, e.g., comprising at least 90 wt % alpha-alumina, at least 95 wt % alpha-alumina, or at least 99 wt % alpha-alumina.

In other embodiments, the support is a silica support, e.g., comprising at least 90 wt % silica, at least 95 wt % silica, or at least 99 wt % alumina. In still other embodiments, the support is a silica-alumina support, e.g., comprising at least 90 wt % silica and alumina, at least 95 wt % silica and alumina, or at least 99 wt % silica and alumina.

The porous support can be provided with a variety of surface areas. For example, in various embodiments of the catalyst compositions of the disclosure, the support has a BET surface area of no more than 10 m²/g. The person of ordinary skill in the art will appreciate that the “BET surface area” of a material refers to the specific surface area of a material, and is determined through the standard testing method ASTM D3663 (“Standard Test Method for Surface Area of Catalysts and Catalyst Carriers”). For example, in some embodiments, the porous support has a BET surface area of no more than 7 m²/g. In some embodiments as otherwise described herein, the porous support has a BET surface area in the range of 1-10 m²/g. For example, in various embodiments, the porous support has a BET surface area in the range of 1-7 m²/g, or 2-10 m²/g, or 2-7 m²/g, or 5-10 m²/g.

Supports suitable for use herein can be provided with a range of pore volumes. In various embodiments of the catalyst compositions as otherwise described herein, the porous support has a pore volume (determined using mercury intrusion porosimetry according to ASTM D4284) of at least 0.1 cm³/g. For example, in some embodiments, the porous support has a pore volume of at least 0.2 cm³/g. In various such embodiments as otherwise described herein, the porous support has a pore volume in the range of 0.1-1.0 cm³/g. For example, in various embodiments, the porous support has a pore volume in the range of 0.1-0.8 cm³/g, or 0.1-0.6 cm³/g, or 0.1-0.5 cm³/g, or 0.2-1.0 cm³/g, or 0.2-0.8 cm³/g, or 0.2-0.6 cm³/g, or 0.2-0.5 cm³/g.

The person of ordinary skill can provide suitable porous supports for use herein. The average pore size is not particularly limited and the person of ordinary skill in the art will select a pore size distribution appropriate for a desired catalytic process based on the present disclosure. In some embodiments as otherwise described herein, the porous support has a bimodal pore size distribution, with a first mode having a peak in the range of 3-20 nm and a second peak in the range of 50-200 nm, as measured by mercury intrusion porosimetry according to ASTM D4284. In various embodiments, the porous support has at least 90% of its pore volume in pores in the range of 3-200 nm as measured by mercury intrusion porosimetry according to ASTM D4284. As the person of ordinary skill in the art will appreciate, the mercury intrusion porosimetry technique is limited in its range; for example, it will not measure macropores. In various embodiments, no more than 20% (e.g., no more than 10%, or no more than 5%) of the volume of the catalyst composition (i.e., within the outer surfaces of catalyst pieces) is made up of macropores (i.e., internal pores having a dimension greater than 1000 nm). But the person of ordinary skill in the art will appreciate that other pore size distributions can be suitable.

The supports can be provided in a number of forms. In various embodiments, as otherwise described herein, the catalyst is provided as a plurality of pieces, each having a support having a volume in the range of 0.5 mm³ to 1000 mm³ (e.g., 1 mm³-500 mm³). Such supports can be formed in a variety of shapes, e.g., cylindrical, spherical, ovoidal, toroidal, parallepipedal, or multilobal; ends can be chamfered or otherwise shaped as desirable. Various such supports can be made, e.g., by extrusion, tableting, spray drying.

As described above, the active catalytic species can be provided in relatively small amounts. Accordingly, the supported catalyst compositions of the disclosure can have relatively high proportions of support. For example, in various embodiments, the porous support is present in the catalyst in an amount of at least 95 wt %, e.g., at least 97 wt %, at least 98 wt %, or at least 99 wt %. Amounts of support are calculated on an as-calcined basis, as the most stable oxide(s) as a fraction of calcined catalyst composition weight.

As described above, the catalyst composition includes palladium in an amount of at least 0.005 wt %. The form of the palladium is not particularly limited and may be present in the catalyst in a variety of forms at a variety of times during the life of the catalyst composition. Typically, the palladium is in the form of an oxide and/or metallic palladium. For example, upon calcining, palladium is typically primarily present in the form of an oxide. The palladium is typically at least partially reduced (by contact with a reductant such has hydrogen) before catalytic use; in its active catalyst form, there is typically a combination of Pd(0) and palladium oxide present. Of course, other forms are possible (e.g., a palladium salt). For purposes of this disclosure, the amount of palladium present is calculated as the weight percentage of palladium in the catalyst based on the total weight of the catalyst composition, calculated as Pd(0) regardless of the form in which the palladium may be present. In various embodiments as otherwise described herein, the palladium is present in an amount in the range of at least 0.007 wt %, or at least 0.01 wt %, or at least 0.015 wt %. The present inventors have noted that while a variety of amounts of palladium can be used, good catalytic effect is observed with relatively small amounts. Thus, given the expense of palladium, it can be desirable to use only a limited amount. For example, in some embodiments of the disclosure as otherwise described herein, the palladium is present in an amount in the range of 0.005 wt % to 0.2 wt %, e.g., 0.005-0.1 wt %, or, or 0.005-0.05 wt %, or 0.005-0.02 wt %, based on the weight of the catalyst composition. In some embodiments, the palladium is present in an amount in the range of 0.007 wt % to 0.2 wt %, e.g., 0.007-0.1 wt %, or 0.007-0.05 wt %, or 0.007-0.02 wt %, based on the weight of the catalyst composition. In various embodiments, the palladium is present in an amount in the range of 0.01 to 0.2 wt %, e.g., 0.01 to 0.1 wt %, or 0.01 to 0.05 wt %, or 0.01 to 0.02 wt %, based on the weight of the catalyst composition. In some embodiments, the palladium is present in an amount in the range of 0.015 wt % to 0.2 wt %, e.g., 0.015 to 0.1 wt %, or 0.015 to 0.05 wt %, or 0.015 to 0.03 wt %, based on the weight of the catalyst composition. Of course, more palladium may be used, but in many such cases no further improvement in catalytic activity may be achieved.

As described below, the person of ordinary skill in the art can use impregnation methods such as incipient wetness impregnation to provide palladium to the catalyst composition.

As described above, the catalyst composition also includes cesium in an amount of at least 0.01 wt %. The present inventors have determined that inclusion of cesium in the catalyst can provide improved performance, especially with respect to selectivity and stability to runaway, as described with reference to the Examples below. The form of cesium is not particularly limited and may be present in the catalyst composition in a variety of forms at a variety of times during the life of the catalyst composition. Typically, the cesium is in the form of an oxide after calcination. For example, upon calcining, palladium is typically primarily present in the form of an oxide. Without intending to be bound by theory, the inventors surmise that when the palladium is at least partially reduced before catalytic use, the cesium primarily remains as oxide, but some cesium(0) may be formed. Of course, other forms are possible (e.g., a cesium salt). For purposes of this disclosure, the amount of cesium present is calculated as the weight percentage of the cesium atoms in the catalyst composition based on the total weight of the catalyst composition, calculated as Cs(0), regardless of the form in which cesium may be present. In various embodiments, the cesium is present in an amount of at least 0.02 wt %, e.g., at least 0.05 wt %, at least 0.07 wt %, or at least 0.1 wt %. In various embodiments, the cesium is present in an amount in the range of 0.01 to 2 wt %, e.g., 0.01 to 1.5 wt %, or 0.01 to 1 wt %, or 0.01 to 0.5 wt %, or 0.01 to 0.3 wt %, or 0.01 to 0.1 wt %, based on the weight of the catalyst composition. In various embodiments, the cesium is present in the range of 0.02 to 2 wt %, e.g., 0.02 to 1.5 wt %, or 0.2 to 1 wt %, or 0.02 to 0.5 wt %, or 0.02 to 0.3 wt %, or 0.02 to 0.1 wt %, based on the weight of the catalyst composition. In various embodiments, the cesium is present in an amount in the range of 0.05 to 2 wt %, e.g., 0.05 to 1.5 wt %, or 0.05 to 1 wt %, or 0.05 to 0.5 wt %, or 0.05 to 0.3 wt %, or 0.05 to 0.15 wt % based on the weight of the catalyst composition. In various embodiments, the cesium is present in an amount in the range of 0.07 to 2 wt %, e.g., 0.07 to 0.5 wt %, or 0.07 to 0.3 wt %, or 0.07 to 0.2 wt %, based on the weight of the catalyst composition. In various embodiments, the cesium is present in an amount in the range of 0.1 to 2 wt %, e.g., 0.1 to 1.5 wt %, or 0.1 to 1 wt %, or 0.1 to 0.5 wt %, based on the weight of the catalyst composition.

As described below, the person of ordinary skill in the art can use impregnation methods such as incipient wetness impregnation to provide cesium to the catalyst composition.

In various desirable embodiments, at least as much cesium as palladium is present in the catalyst composition, i.e., on a weight basis. For example, in various embodiments, the cesium is present in an amount of at least 1.5 times that of palladium (i.e., on a weight basis). In various embodiments, the amount of cesium is at least 2 times, 2.5 times, at least 3 times, or even at least 3.5 times the amount of palladium.

As described above, the catalyst composition can optionally include a metallic modifier selected from silver, gold, zinc, tin, lead, cadmium, bismuth, gallium, and copper. As with palladium and cesium, the metallic modifier may be present in a variety of forms at a variety of times during catalyst lifetime, for example, oxide (especially immediately after calcining); metal (e.g., after reduction); or salt, or a combination thereof.

In various especially desirable embodiments, the metallic modifier is silver. The present inventors have determined specifically that the inclusion of silver as the metallic modifier provides additional improvement in performances of the catalyst composition as otherwise described herein.

For purposes of this disclosure, the amount of metallic modifier present is calculated as the weight percentage of metallic modifier in the catalyst based on the total weight of the catalyst composition, calculated as M(0), regardless of the form in which the metallic modifier may be present. In various embodiments, the metallic promoter is present in an amount of up to about 0.5 wt % based on the weight of the catalyst composition. For example, in various embodiments, if present, the metallic modifier is present in an amount of up to about 0.2 wt %, or up to about 0.1 wt %, based on the weight of the catalyst composition. In various embodiments of the catalyst compositions as otherwise described herein, the metallic modifier is present in an amount in the range of 0.01 wt % to 0.5 wt % based on the weight of the catalyst composition. For example, in various embodiments, the metallic modifier is in the range of 0.01 wt % to 0.2 wt %, or 0.01 to 0.1 wt % based on the weight of the catalyst composition. In some embodiments, the metallic modifier is present in an amount in the range of 0.02 wt % to 0.5 wt % based on the weight of the catalyst composition. For example, in various embodiments, if present, the metallic modifier is present in an amount in the range of 0.02 to 0.2 wt %, or 0.02 to 0.1 wt % based on the weight of the catalyst composition.

The metallic modifier and palladium can be provided to the catalyst composition in a variety of weight ratios. For example, in some embodiments, the weight ratio of the metallic promoter, if present, to the palladium is at least 1:1. In various embodiments, the weight ratio of the metallic promoter, if present, to the palladium is at least 1.5:1, or at least 2:1. In some embodiments, the weight ratio of the metallic promoter, if present, to the palladium is at most about 12:1. For example, in various embodiments, the weight ratio of the metallic promoter, if present, to the palladium is at most 10:1 or 5:1. In some embodiments, the weight ratio of the metallic promoter, if present, to the palladium is in the range of 1:1 to 12:1. For example, in various embodiments, the weight ratio of the metallic promoter, if present, to the palladium is in the range of 1:1 to 12:1, or 1:1 to 10:1, or 1:1 to 5:1, or 1.5:1 to 12:1, or 1.5:1 to 10:1, or 1.5:1 to 5:1, or 2:1, to 12:1, or 2:1 to 10:1, or 2:1 to 5:1.

In various embodiments as otherwise described herein, palladium is localized at the outer surface of the catalyst, e.g., in a so-called shell catalyst configuration. Materials “localized at an outer surface” have a substantially higher concentration (e.g., at least 100% higher) at the outer surface of the material than in the interior of the material. As used herein, an outer surface is a surface at the overall envelope of the catalyst material, e.g., in the shape of a pellet; it does not include a surface of a pore that is interior to the overall envelope of the material shape. The person of ordinary skill in the art will further appreciate that the “outer surface” of a composition does not consist solely of the outermost surface of atoms of a composition, but rather includes a surface layer at the outside of the composition.

For example, in various embodiments of the present disclosure as otherwise described herein, at least 80 wt % (e.g., at least 90 wt %) of the palladium is within 1000 microns of an outer surface of the porous support. In various embodiments as otherwise described herein, at least 80 wt % (e.g., at least 90 wt %) of the palladium is within 800 microns of an outer surface of the porous support. In various embodiments as otherwise described herein, at least 80 wt % (e.g., at least 90 wt %) of the palladium is within 500 microns of an outer surface of the porous support.

In various embodiments, the cesium and/or the metallic modifier (when present) is dispersed substantially throughout the support. Thus, in some embodiments, while the palladium is localized at the surface of the support, the cesium and/or metallic modifier are dispersed substantially throughout the support. Without intending to be bound by theory, the present inventors note that many metallic modifiers, like silver, and alkali species, like cesium, are relatively mobile during processing, and can diffuse throughout the support, e.g., during calcining. Palladium is much less mobile, and can tend to remain in place during processing. Moreover, catalyst synthesis techniques can be performed to help provide such a configuration. For example, this configuration can be accomplished by impregnating the cesium and/or metallic modifier in a first impregnation step that impregnates throughout the support, and impregnating the palladium using incipient wetness or some other technique that impregnates only to a desired depth. And, of course, other configurations are possible.

In various embodiments, the catalyst composition of the present disclosure as otherwise described herein includes an alpha-alumina support, present in an amount of at least 98 wt %; palladium, present in an amount in the range of 0.01 wt % to 0.1 wt % (e.g., 0.01 wt % to 0.05 wt %) based on the weight of the catalyst; cesium, present in an amount of at least 2.5 times that of palladium; and silver, present in an amount of at least 2 times that of palladium. For example, in some embodiments, cesium is present in an amount in the range of 0.02 wt % to 2 wt % based on the weight of the catalyst; in some embodiments, silver is present in an amount in the range of 0.02 wt % to 0.5 wt % based on the weight of the catalyst.

In various embodiments, the catalyst composition of the present disclosure as otherwise described herein includes an alpha-alumina support, present in an amount of at least 98 wt %; palladium, present in an amount in the range of 0.01 wt % to 0.1 wt % (e.g., 0.01 wt % to 0.05 wt %), based on the weight of the catalyst; cesium, present in an amount of at least 1.5 times that of palladium; and silver, present in an amount of at least 1.5 times that of palladium; wherein the cesium and silver are substantially distributed (e.g., homogeneously) throughout the support; and wherein at least 80 wt % (e.g., at least 90 wt %) of the palladium is within 1000 microns of an outer surface of the porous support. For example, in some embodiments, cesium is present in an amount in the range of 0.02 wt % to 2 wt % based on the weight of the catalyst; in some embodiments, silver is present in an amount in the range of 0.02 wt % to 0.15 wt % based on the weight of the catalyst.

The person of ordinary skill in the art will appreciate that the catalysts of the disclosure can be provided in many forms, depending especially on the particular form of the reactor system in which they are to be used, e.g., in a fixed bed or as a fluid bed. The catalysts can be provided themselves as discrete bodies of material, e.g., as porous particles, pellets or shaped extrudates. In various embodiments, as otherwise described herein, the catalyst composition is provided as a plurality of pieces, each having a support having a volume in the range of 0.5 mm³ to 1000 mm³ (e.g., 1 mm³-500 mm³). The pieces can be provided in a number of forms, e.g., cylindrical, spherical, ovoidal, toroidal, parallepipedal, or multilobal; ends can be chamfered or otherwise shaped as desirable. However, in other embodiments, a catalyst of the disclosure can itself be formed as a layer on an underlying substrate. The underlying substrate is not particularly limited. It can be formed of, e.g., a metal or metal oxide, and can itself be provided in a number of forms, such as particles, pellets or shaped extrudates, e.g., as described above.

The catalyst composition can be provided with a variety of surface areas. For example, in various embodiments of the catalyst compositions of the disclosure, the overall catalyst has BET surface area of no more than 10 m²/g. The person of ordinary skill in the art will appreciate that the “BET surface area” of a material refers to the specific surface area of a material, and is determined through the standard testing method ASTM D3663 (“Standard Test Method for Surface Area of Catalysts and Catalyst Carriers”). For example, in some embodiments, the catalyst composition has a BET surface area of no more than 7 m²/g. In some embodiments as otherwise described herein, the catalyst composition has a BET surface area in the range of 1-10 m²/g. For example, in various embodiments, the catalyst composition has a BET surface area in the range of 1-7 m²/g, or 2-10 m²/g, or 2-7 m²/g, or 5-10 m²/g.

Catalyst compositions suitable for use herein can be provided with a range of pore volumes. In various embodiments, a catalyst composition as otherwise described herein has a pore volume (determined using mercury intrusion porosimetry according to ASTM D4284) of at least 0.1 cm³/g. For example, in some embodiments, the catalyst composition has a pore volume of at least 0.2 cm³/g. In various such embodiments as otherwise described herein, the catalyst composition has a pore volume in the range of 0.1-1.0 cm³/g. For example, in various embodiments, the catalyst composition has a pore volume in the range of 0.1-0.8 cm³/g, or 0.1-0.6 cm³/g, or 0.1-0.5 cm³/g, or 0.2-1.0 cm³/g, or 0.2-0.8 cm³/g, or 0.2-0.6 cm³/g, or 0.2-0.5 cm³/g.

In some embodiments as otherwise described herein, the catalyst composition has a bimodal pore size distribution, with a first mode having a peak in the range of 3-20 nm and a second peak in the range of 50-200 nm, as measured by mercury intrusion porosimetry according to ASTM D4284. In various embodiments, the catalyst composition has at least 90% of its pore volume in pores in the range of 3-200 nm as measured by mercury intrusion porosimetry according to ASTM D4284. But the person of ordinary skill in the art will appreciate that other pore size distributions can be suitable.

Another aspect of the present disclosure provides a method of making a catalyst composition. As described above, the method of making a catalyst composition includes providing a porous support; contacting the porous support with a first solution comprising a palladium compound and optionally a metallic compound; drying the porous support to provide a palladium impregnated support; contacting the palladium impregnated support with a second solution comprising a cesium compound; and drying and calcining the palladium impregnated support to provide the catalyst composition.

Supports suitable for methods as otherwise described herein can be provided with a range of BET surface areas, pore volumes, and pore sizes. In some embodiments, the method includes providing a porous support having a BET surface area of no more than 10 m²/g. In some embodiments of the present disclosure, the porous support has a BET surface area of no more than 7 m²/g. For example, in various embodiments, the porous support has a BET surface area in the range of 1-10 m²/g, or 1-9 m²/g, or 1-8 m²/g, or 1-7 m²/g, or 2-10 m²/g, or 2-9 m²/g, or 2-8 m²/g, or 2-7 m²/g, or 3-10 m²/g, or 3-9 m²/g, or 3-8 m²/g, or 3-7 m²/g.

In some embodiments, the method includes providing a porous support having a pore volume of at least 0.1 cm³/g. In some embodiments, the porous support has a pore volume of at least 0.2 cm³/g. For example, in various embodiments, the porous support has a pore volume in the range of 0.1-0.8 cm³/g, or 0.1-0.7 cm³/g, or 0.1-0.6 cm³/g, or 0.1-0.5 cm³/g, or 0.2-0.8 cm³/g, or 0.2-0.7 cm³/g, or 0.2-0.6 cm³/g, or 0.2-0.5 cm³/g.

In various embodiments, the method includes providing a support as described above.

As described above, the method of making a catalyst composition includes contacting the porous support with a first solution comprising a palladium compound and optionally a metallic compound. In some embodiments, the first solution comprises a palladium compound, a first solvent, and optionally a metallic compound. In some embodiments of the present disclosure as otherwise described herein, the palladium compound is a palladium salt. For example, in various embodiments, the palladium salt is independently selected from palladium nitrate, palladium phosphate, palladium acetate, or palladium sulfate. In some embodiments of the present disclosure as otherwise described herein, the metallic compound, if present, is a metallic salt. For example, in various embodiments, the metallic salt is independently selected from a silver salt, a gold salt, a zinc slat, a tin salt, a lead salt, a cadmium salt, a bismuth salt, a gallium salt, and a copper salt. In some embodiments, the metallic compound, if present, is a silver salt. For example, in various embodiments, the silver salt is independently selected from silver nitrate, silver phosphate, silver acetate, or silver sulfate. In some embodiments, the first solvent is water. As the person of ordinary skill in the art will appreciate, these metal species are conveniently provided in the same first solution, so that only one step of contacting the support with solution is required. However, other schemes are possible. Additionally, the person of ordinary skill in the art will understand that the amount of palladium compound and optional metallic compound in the first solution can vary based on the desired loading of the support.

In some embodiments, the first solution further comprises an acid. For example, in various embodiments, the acid is independently selected from nitric acid, phosphoric acid, acetic acid, or sulfuric acid.

As described above, the method includes contacting the porous support with the first solution. As the person of ordinary skill in the art would appreciate, the time and temperature at which contacting occurs depends on the desired loading of the support and the physical characteristics of the support itself (e.g., pore volume, pore size, etc.). As such, the person of ordinary skill in the art would be able to choose an appropriate time and temperature to contact the porous support with the first solution. For example, in some embodiments, contacting the porous support with the first solution occurs for a time in the range of 30 minutes to 3 hours. In various embodiments, the time is in the range of 30 minutes to 2 hours, or 30 minutes to 1 hour, or 1 hour to 3 hours, or 1 hour to 2 hours. In some embodiments, contacting the porous support with the first solution occurs at an ambient temperature.

As described above, the method includes drying and calcining the porous support to provide a palladium impregnated support. In some embodiments, calcining the porous support occurs at a temperature in the range of 450° C. to 650° C. For example, in various embodiments, drying the porous support occurs at a temperature in the range of 450 to 600° C., or 450 to 550° C., or 500 to 650° C., or 500 to 600° C., or 500 to 550° C. In some embodiments, calcining the porous support occurs for a time in the range of 1 hour to 4 hours. For example, in some embodiments, drying the porous support occurs for time in the range of 1 to 3 hours, or 1 to 2 hours, or 2 to 4 hours, or 2 to 3 hours.

The method includes contacting the palladium-impregnated support with a second solution comprising a cesium compound. For example, in some embodiments, the cesium compound is cesium hydroxide. In some embodiments of the present disclosure as otherwise described herein, the second solution comprises a cesium compound and a second solvent. For example, in some embodiments, the second solvent is water. In some embodiments of the present disclosure, the second solution does not comprise a reducing agent. For example, in various embodiments, the second solution does not include an alkali metal borohydride, hydrazine, formaldehyde, formic acid, ascorbic acid, dextrose, or aluminum powder. The person of ordinary skill in the art will appreciate that the amount of cesium compounds in the second solution depends on the desired loading of the support.

As described above, the method includes contacting the palladium-impregnated catalyst with the second solution. As the person of ordinary skill in the art would appreciate, the time and temperature at which contacting occurs depends on the desired loading of the support and the physical characteristics of the support itself (e.g., pore volume, pore size, etc.). In some embodiments, contacting the palladium-impregnated support with the second solution occurs for a time in the range of 20 minutes to 3 hours. In various embodiments, contacting the palladium-impregnated support with the second solution occurs for a time in the range of 30 minutes to 2 hours, or 30 minutes to 1 hour, or 1 hour to 3 hours, or 1 hour to 2 hours. In some embodiments, contacting the palladium impregnated support with the second solution occurs at an ambient temperature.

As described above, the method includes drying and calcining the palladium impregnated support to provide the catalyst composition. In some embodiments, calcining the palladium-impregnated support occurs at a temperature in the range of 350° C. to 550° C. For example, the temperature may be in the range of 350 to 500° C., or 350 to 450° C., or 400 to 550° C., or 400 to 500° C. In some embodiments, calcining the palladium-impregnated support occurs at a time in the range of 1 hour to 4 hours. For example, in various embodiments, the time is in the range of 1 to 3 hours, or 1 to 2 hours, or 2 to 4 hours, or 2 to 3 hours.

In some embodiments of the method as otherwise described herein, the catalyst composition comprises a porous support, present in an amount of at least 98 wt %; palladium, present in an amount in the range of 0.01 wt % to 0.05 wt % based on the weight of the catalyst composition; cesium, present in an amount in the range of 0.02 wt % to 0.6 wt % based on the weight of the catalyst composition; and optionally, a metallic promoter, wherein if present is in an amount of up to about 0.2 wt % based on the weight of the catalyst. Another aspect of the present disclosure provides a catalyst composition as otherwise described herein made by the method as otherwise described herein.

Notably, the catalyst compositions described here can be provided and loaded into a reactor in a calcined form. Accordingly, the catalyst compositions described herein can be provided in substantially oxidic form, which makes them relatively stable to packaging, transport and storage.

Thus, in various embodiments as otherwise described herein, no more than 25 mol %, e.g., no more than 10 mol % or no more than 5 mol %, of the palladium is present as Pd(0). In various embodiments as otherwise described herein, at least 75 mol %, for example, at least 90 mol % or at least 95 mol % of the palladium is in the form of one or more palladium oxides and/or hydroxides (e.g., including PdO).

In various embodiments as otherwise described herein, no more than 25 mol %, e.g., no more than 10 mol % or no more than 5 mol %, of the silver is present as Ag(0). In various embodiments as otherwise described herein, at least 75 mol %, for example, at least 90 mol % or at least 95 mol % of the silver is in the form of one or more silver oxides and/or hydroxides (e.g., including Ag₂O and/or AgO).

In various embodiments as otherwise described herein, no more than 25 mol %, e.g., no more than 10 mol % or no more than 5 mol %, of the cesium is present as Cs(0). In various embodiments as otherwise described herein, at least 75 mol %, for example, at least 90 mol % or at least 95 mol % of the cesium is in the form of one or more cesium oxides and/or hydroxides (e.g., including Cs₂O).

Another aspect of the present disclosure provides a method of selective hydrogenation of alkyne (e.g., acetylene or methylacetylene) in an olefin feed stream comprising hydrogen and alkyne. As described above, the method includes contacting the olefin feed stream with a catalyst composition as otherwise described here.

The catalyst compositions described herein are especially useful in so-called front-end hydrogenation processes, in which alkynes present in an olefin stream are selectively reduced without substantial reduction of the olefin itself.

In some embodiments, the olefin feed stream comprises C1-C3 hydrocarbons. For example in some embodiments, the olefin feed stream comprises acetylene (C₂H₂) and ethylene. In other embodiments, the olefin feed stream comprises methylacetylene and propylene.

The amounts of materials in the olefin feed stream can vary. For example, in various embodiments, such as in so-called front-end hydrogenation processes, the olefin feed stream comprises at least 10 mol % olefin, e.g., at least 20 mol % olefin. In various such embodiments (e.g., front-end embodiments), there is no more than 70 mol % olefin, e.g., no more than 60 mol % olefin, or no more than 50 mol % olefin in the olefin feed stream. As noted above, the olefin can be, e.g., ethylene, propylene, or a combination thereof.

In various embodiments (e.g., front-end embodiments), the olefin feed stream comprises at least 1 ppm (on a molar basis) alkyne, e.g., at least 10 ppm alkyne, or at least 100 ppm alkyne, or at least 500 ppm alkyne. In various such embodiments (e.g., front-end embodiments), there is no more than 2 mol % alkyne, e.g., no more than 1.5 mol % alkyne, or no more than 1 mol % alkyne in the olefin feed stream. As noted above, the alkyne can be, e.g., acetylene, methylacetylene, or a combination thereof.

In various embodiments (e.g., front-end embodiments), the olefin feed stream comprises at least 5 mol % hydrogen, e.g., at least 10 mol % hydrogen, or at least 15 mol % hydrogen. In various such embodiments (e.g., front-end embodiments), the olefin feed stream comprises no more than 40% hydrogen, e.g., no more than 30% hydrogen, or no more than 20% hydrogen.

As the person of ordinary skill in the art will appreciate, various other components, can be present. For example, in various embodiments, (e.g., front-end embodiments), inert components, such as methane can be present. In various embodiments, (e.g., front-end embodiments), one or more inerts (e.g., methane, ethane, propane, desirably methane) are present in an amount of at least 5 mol %, e.g., at least 10 mol % or at least 20 mol %. In various such embodiments, (e.g., front-end embodiments), no more than 70 mol % of one or more inerts (e.g., methane, ethane, propane, desirably methane), e.g., no more than 60 mol % or no more than 50 mol %, is present in the olefin feed stream.

In so-called back-end or tail-end hydrogenation configurations, there is typically less hydrogen present. Accordingly, for such reactions and other reactions in which sufficient hydrogen is not otherwise present, the feed can be supplemented with addition of hydrogen. In various embodiments, (e.g., back-end or tail-end embodiments), the optionally-supplemented olefin feed stream contains hydrogen in an amount of at least 0.1 mol %, e.g., at least 0.2 mol %, or at least 0.5 mol %, or at least 1 mol %. In some such embodiments, (e.g., back-end or tail-end embodiments), the optionally-supplemented olefin feed stream contains hydrogen in an amount of no more than 20 mol %, e.g., no more than 15 mol %, or no more than 7 mol %, or no more than 5 mol %. The person of ordinary skill in the art can select an amount of hydrogen based on a number of factors, including the amount of alkyne present.

In various embodiments (e.g., back-end/tail-end embodiments) as otherwise described herein, the olefin feed stream includes at least 1 ppm (on a molar basis) alkyne, e.g., at least 10 ppm alkyne, or at least 100 ppm alkyne, or at least 500 ppm alkyne. In various such embodiments (e.g., back-end/tail-end embodiments), there is no more than 2 mol % alkyne, e.g., no more than 1.5 mol % alkyne, or no more than 1 mol % alkyne, in the olefin feed stream. As noted above, the alkyne can be, e.g., acetylene, methylacetylene, or a combination thereof.

In various embodiments (e.g., back-end/tail-end embodiments) as otherwise described herein, the olefin feed stream includes at least 20 mol % olefin, e.g., at least 50 mol % olefin, or at least 70 mol % olefin. In various such embodiments (e.g., back-end/tail-end embodiments), there is no more than 90 mol % olefin, e.g., no more than 80 mol % olefin, or no more than 70 mol % olefin, in the olefin feed stream. As noted above, the olefin can be, e.g., ethylene, propylene, or a combination thereof.

As the person of ordinary skill in the art will appreciate, various other components, can be present. For example, in various embodiments (e.g., back-end/tail-end embodiments), inert components, such as methane can be present. In various embodiments (e.g., back-end/tail-end embodiments), one or more inerts (e.g., methane, ethane, propane, desirably methane) are present in an amount of at least up to 30 mol %, e.g., up to 20 mol % or up to 10 mol %.

In various embodiments, regardless of configuration, the olefin feed stream further comprises carbon monoxide, or carbon monoxide is otherwise provided to the reaction. Carbon monoxide can act to help prevent runaway, as is known in the art. Carbon monoxide can be present in the reactor in a variety of amounts, e.g., up to 1000 ppm, or up to 700 ppm, or up to 500 ppm, or 30-1000 ppm, or 30-700 ppm, or 30-500 ppm, or 60-1000 ppm, or 60-700 ppm, or 60-500 ppm, or 100-1000 ppm, or 100-700 ppm, or 100-500 ppm.

The alkyne of the olefin feed stream is contacted with hydrogen and the catalyst. For example, in various embodiments, the molar ratio of hydrogen to alkyne is in the range of from about 1 to about 1000, or from about 1.1 to about 800.

The operating parameters of the selective hydrogenation of alkyne are not narrowly critical and can be controlled by the person of ordinary skill in the art in view of a number of interrelated factors including, but not limited to, the chemical composition of the feedstock, the control systems and design of the particular plant, etc.

In some embodiments, the contacting with the catalyst composition occurs at a temperature sufficient to catalyze acetylene to ethylene, or methylacetylene to propylene. For example, in various embodiments, the temperature is in the range of 20 to 150° C., or 40 to 150° C., or 60-150° C., or 20 to 130° C., or 40 to 130° C., or 60-130° C., or 20-110° C., or 40 to 110° C., or 60-110° C. In various embodiments, the contacting occurs at a GSHV in the range of 5,000 to 20,000 h⁻¹, or in the range of 5000 h⁻¹ to 15,000 h⁻¹, or 8,000 to 20,000 h⁻¹, or 8000-15,000 h⁻¹. In various embodiments, the catalyzing occurs at a pressure in the range of 100 to 500 psig, or 100 to 400 psig, or 200 to 500 psig, or 200 to 400 psig. Of course, the person of ordinary skill in the art will determine other combinations of temperatures, pressures, and space velocities that can provide good results.

In some embodiments, the method of selective hydrogenation of alkyne provides an output olefin stream having less than 25 ppm alkyne. In some embodiments, the method of selective hydrogenation of alkyne is conducted such that the amount of alkyne in the output olefin stream is no more than 2% of the amount of the corresponding alkene of the olefin feed stream.

As described above, the selectivity and ease of operability of a catalyst for the selective hydrogenation of acetylene depends on the operation window, or the temperature difference between the runaway temperature (T2) and the clean-up temperature (T1). The present inventors have found a method of selective hydrogenation of alkyne that provides a relatively large operation window. For example, in various embodiments, the method of selective hydrogenation of alkyne has an operating window of at least 10 degrees, or at least 15 degrees, or at least 20 degrees, or at least 25 degrees, or at least 30 degrees, or at least 35 degrees.

In various embodiments, the methods include loading the catalyst composition in a reactor; the contacting of the alkyne and hydrogen with the catalyst composition occurs in the reactor. The form of the reactor is not particularly limited, and the person of ordinary skill in the art can adapt any desirable hydrogenation reactor system to perform the methods described herein.

Notably, in various desirable embodiments, the catalyst composition need not be pre-reduced before being loaded into the reactor. Thus, the catalyst composition can be in a substantially oxidic form and/or have relatively little reduced metal (e.g., in any manner as described above with respect to catalyst compositions) at the time that it is loaded into the reactor.

As the person of ordinary skill in the art will appreciate, and as is conventional for palladium-based hydrogenation catalysts, it is desirable that the palladium be in a substantially reduced state for catalytic activity. In many configurations, such as in front-end configurations, this can occur in the presence of hydrogen and/or CO in the olefin feed stream, and so no separate formal reduction step is necessary. In such cases, however, there may be an induction period during which substantial hydrogenation does not occur. In other configurations, e.g., back-end or tail-end configurations, there is relatively little hydrogen present and it may be desirable to reduce the catalyst in a separate step. The catalyst composition can be activated by contact with hydrogen at an elevated temperature. Without intending to be bound by theory, it is believed that this substantially converts the palladium to Pd(0), which is believed to the be the active catalytic species in the hydrogenation. For example, contact with a stream of hydrogen in nitrogen carrier (at least 1 vol %) at a temperature in the range of 50-200 C can be used to activate the catalyst. Activation processes for such catalysts are well-known and the person of ordinary skill in the art can adapt such processes for use here.

In various aspects and embodiments, the methods as otherwise described herein can be conducted in a selective hydrogenation reactor (or reactors) housing a catalyst bed or a series of catalyst beds containing a catalyst composition (e.g., a catalyst composition as otherwise described herein) capable of selectively hydrogenating alkynes.

The catalysts according to these aspects of the disclosure can otherwise be as described above with respect to catalyst compositions useful in the methods of the disclosure. Moreover, the catalyst compositions according to this aspect of the disclosure can be used in any of the methods as otherwise described herein.

Notably, as described above, in many embodiments the catalysts described herein do no require pre-reduction before they are loaded into a reactor.

The processes and materials as otherwise described herein can be especially useful in front-end applications. However, the person of ordinary skill in the art will appreciate that they can be used in a variety of other applications, especially those in which risk of runaway (e.g., due to high hydrogen concentrations) is problematic. Thus, they can be useful in so-called back-end or tail-end hydrogenations as well.

### EXAMPLES

The Examples that follow are illustrative of specific embodiments of the process of the disclosure, and various uses thereof. They are set forth for explanatory purposes only, and are not to be taken as limiting the scope of the disclosure.

### Example 1. Catalyst Preparation

Cesium based catalysts of the present invention were prepared to evaluate their performance for hydrogenation of acetylene.

Catalyst A, a first control catalyst without cesium, was prepared by incipient wetness impregnation (IWI), with a nitric acid solution of palladium nitrate and an alumina carrier (4 mm×4 mm tablet), having a surface area of 6 m²/g, so as to obtain a final catalyst having 0.02 wt % palladium. After impregnation sitting for 1 hour, the catalyst precursor was calcined at 550° C. for 2 hours. X-ray fluorescence (XRF) results showed that palladium was present at 0.0199 wt % in the final catalyst.

Catalyst B, with cesium but without silver, was obtained by impregnating, with IWI, a CsOH solution into Catalyst A to achieve 0.2 wt % Cs in the final catalyst. After impregnation, the catalyst precursor was calcined at 460° C. for 2 hours to provide the final catalyst.

Catalyst C, a second control catalyst without cesium, was prepared by an incipient wetness co-impregnation method. A solution of palladium nitrate and silver nitrate was contacted with an alumina carrier (4 mm×4 mm) to achieve a final catalyst having 0.02 wt % palladium and 0.05 wt % silver. After impregnation sitting for 1 hour, the catalyst precursor was calcined at 550° C. for 2 hours to provide the final catalyst. XRF results showed that palladium was present at 0.0215 wt % and silver was present at 0.054 wt % in the final catalyst.

Catalysts D to H, with cesium, were obtained by impregnating, with incipient wetness impregnation, a CsOH solution into catalyst C to achieve from 0.05 wt % to 0.25 wt % in the final catalyst, followed by calcining at 460° C. for 2 hours. Table 1 reports the composition of catalysts A-H, as determined by XRF.

TABLE 1

| Catalyst Name | Pd (wt %) | Ag (wt %) | Cs (wt %) |
|---|---|---|---|
| A | 0.0199 | 0 | 0 |
| B | 0.0199 | 0 | 0.2 |
| C | 0.0215 | 0.054 | 0 |
| D | 0.0215 | 0.054 | 0.05 |
| E | 0.0215 | 0.054 | 0.1 |
| F | 0.0215 | 0.054 | 0.15 |
| G | 0.0215 | 0.054 | 0.2 |
| H | 0.0215 | 0.054 | 0.25 |

Catalysts I-L, with and without other alkali metals, were obtained by impregnating, with incipient wetness impregnation an alkali metal solution into catalyst C followed by calcining at 460° C. for two hours to obtain alkali metal impregnated catalysts with the palladium and silver. Table 2 reports the compositions of catalysts I-L, as determined by XRF.

TABLE 2

| Catalyst Name | Alkali metal | Pd (wt %) | Ag (wt %) | Alkali (wt %) |
|---|---|---|---|---|
| I | — | 0.0210 | 0.0524 | — |
| J | Li | 0.0210 | 0.0524 | 0.0104 |
| K | Na | 0.0210 | 0.0524 | 0.0346 |
| L | K | 0.0210 | 0.0524 | 0.0586 |

Catalyst M was prepared by the same way as Catalyst C but to achieve a final catalyst having 0.2 wt % palladium and 0.5 wt % silver. After impregnation sitting for 1 hour, the catalyst precursor was calcined at 550° C. for 2 hours to provide the final catalyst. XRF results showed that palladium was present at 0.2 wt % and silver was present at 0.5 wt % in the final catalyst.

Catalysts N, with cesium, was obtained by impregnating, with incipient wetness impregnation, a CsOH solution into Catalyst M to achieve 2 wt % cesium in the final catalyst, followed by calcining at 460° C. for 2 hours. XRF results showed that palladium was present at 0.2 wt %, silver was present at 0.5 wt %, and cesium was present at 2 wt % in the final catalyst.

### Example 2. Catalyst Characterization

The catalysts are further characterized to determine how the metals are distributed in the catalyst. Catalyst C was first measured with a microscope to determine the palladium penetration in the support and Catalyst G was measured with scanning electron microscopy with energy dispersive x-ray analysis (SEM-EDX) to determine the distribution of the cesium in the support.

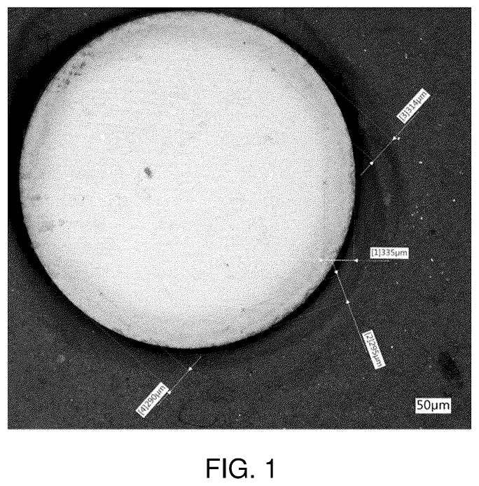

To measure Catalyst C, the catalyst tablet was cut in half, then soaked in a diluted solution of hydrazine. FIG. 1 is a microscope image of the catalyst. The image of FIG. 1 shows the shell structure of the catalyst and that the palladium penetration was an average 298 μm (+34 μm) away from the outer shell, with a minimum penetration of 216 μm and a maximum penetration of 395 μm of palladium.

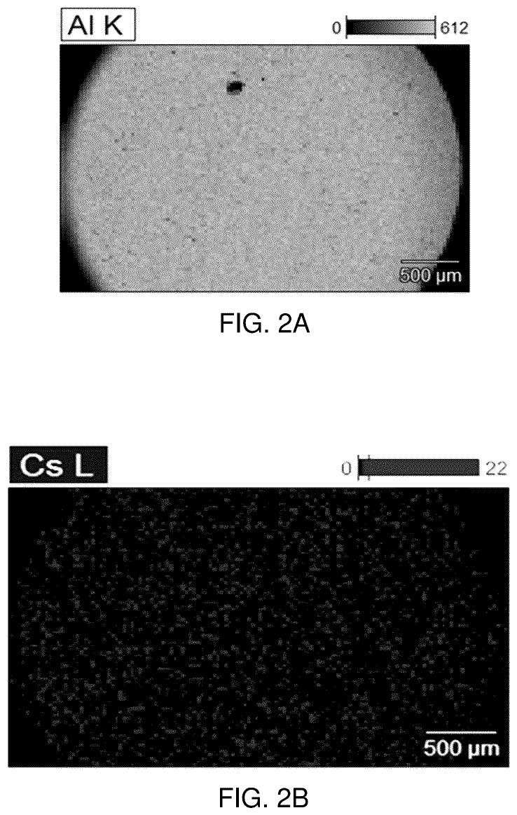

To measure Catalyst G, the catalyst tablet was cut in half and then measured with SEM-EDX to evaluate how the cesium is distributed through the whole support particle. FIG. 2A show the SEM-EDX images of Catalyst G. The image of FIG. 2B shows that cesium is homogeneously distributed from the support skin to the center. It is noted that the palladium and silver loadings are too low for detection by this method (the detection limits for EDS is 2000-5000 ppm).

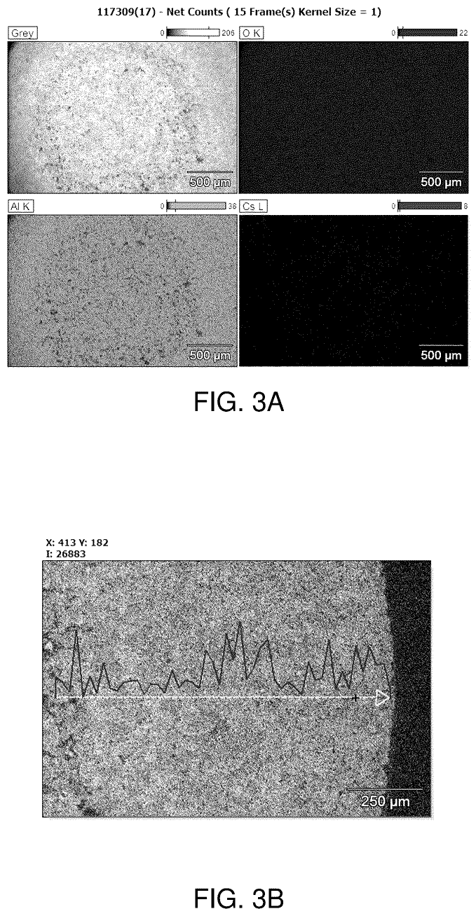

To measure Catalyst N, the catalyst tablet was cut in half and then measured with SEM-EDX to evaluate how the cesium is distributed through the whole support particle. FIG. 3A show the SEM-EDX images of Catalyst N. The image of FIG. 3B shows that cesium is homogeneously distributed from the support skin to the center. It is noted that the palladium and silver loadings are too low for detection by this method (the detection limits for EDS is 2000-5000 ppm).

### Example 3. Catalyst Performance

Catalysts A to M were tested in a lab reactor (with 1.9 cm internal diameter) at a pressure of 3.5 MPa, a GHSV of 7000 h⁻¹ for their effectiveness at selective hydrogenation of acetylene. The feed stream included 20 mol % of H₂, 200 ppm of CO, and 3500 ppm of C₂H₂. Temperature 1 (T1) is the clean-up temperature when the acetylene concentration is 25 ppm in outlet, and Temperature 2 (T2) is the runaway temperature when the ethane concentration is 2 wt % in outlet. The operation window (OW) is defined as difference between T1 and T2. Selectivity is the selectivity to ethylene production at T1. The results of these results are shown in Table 3.

TABLE 3

| Catalyst Name | T1 (° C.) | OW (° C.) | Selectivity at T1 (%) |
|---|---|---|---|
| A | 57 | 8 | 42 |
| B | 71 | 33 | 68 |
| C | 50 | 18 | 57 |
| D | 57 | 29 | 67 |
| E | 59 | 33 | 74 |
| F | 58 | 37 | 76 |
| G | 60 | 38 | 79 |
| H | 60 | 38 | 80 |
| I | 50 | 18 | 57 |
| J | 56 | 21 | 50 |
| K | 53 | 21 | 60 |
| L | 61 | 28 | 67 |

The control catalyst, Catalyst A, showed poor selectivity and a narrow operation window. The addition of cesium significantly improves both the operation windows and selectivity, as shown with the results of Catalyst B. The results demonstrate the strong promotion effect provided by the cesium. Notably, the effect can be achieved by adding the cesium at room temperature without the presence of a wet reducing agent. Further, the catalysts with the addition of both cesium and silver (catalysts D-H) further improves the operation window and selectivity. Additionally, the same promotion effect provided by the cesium is not seen with other alkali metals as shown with the results of catalysts J-L.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparatuses, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the invention (especially in the context of the following claims) are to be construed to cover both the singular and the plural, unless otherwise indicated herein or clearly contradicted by context. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as,” “e.g.,” or “for example”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range within normal ranges of uncertainty and imprecision in the art.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Various aspects and embodiments of the disclosure are provided by the following enumerated embodiments, which may be combined in any number and in any combination not technically or logically inconsistent.

Embodiment 1. A catalyst composition comprising:
a porous support; and, disposed on the support:
palladium, present in an amount of at least 0.005 wt %, e.g., in the range of 0.005 wt % to 0.2 wt %, based on the weight of the catalyst composition; and
cesium, present in an amount of at least 0.01 wt %, e.g., in the range of 0.01 wt % to 2 wt % based on the weight of the catalyst composition.

Embodiment 2. The catalyst composition of embodiment 1, wherein the porous support is an alumina support, e.g., comprising at least 90 wt % alumina, at least 95 wt % alumina, or at least 99 wt % alumina.

Embodiment 3. The catalyst composition of embodiment 1, wherein the porous support is an alpha-alumina support, e.g., comprising at least 90 wt % alpha-alumina, at least 95 wt % alpha-alumina, or at least 99 wt % alpha-alumina.

Embodiment 4. The catalyst composition of embodiment 1, wherein the porous support is a support formed of alumina, silica, titania, or any mixture thereof, e.g., comprising at least 90 wt %, at least 95 wt % or at least 99 wt % of one or more of alumina, silica and titania.

Embodiment 5. The catalyst composition of embodiment 1, wherein the porous support is a silica support, e.g., comprising at least 90 wt % silica, at least 95 wt % silica, or at least 99 wt % silica

Embodiment 6. The catalyst composition of embodiment 1, wherein the porous support is a silica-alumina support, e.g., comprising at least 90 wt % silica and alumina, at least 95 wt % silica and alumina, or at least 99 wt % silica and alumina.

Embodiment 7. The catalyst composition of any of embodiments 1-6, wherein the porous support has a BET surface area of no more than no more than 7 m²/g.

Embodiment 8. The catalyst composition of any of embodiments 1-6, wherein the porous support has a BET surface area in the range of 1-10 m²/g (e.g., in the range of 1-7 m²/g, or 2-10 m²/g, or 2-7 m²/g, or 5-10 m²/g).

Embodiment 9. The catalyst composition of any of embodiments 1-8, wherein the porous support has a pore volume of at least 0.1 cm³/g, e.g., at least 0.2 cm³/g.

Embodiment 10. The catalyst composition of any of embodiments 1-8, wherein the porous support has a pore volume in the range of 0.1-1.0 cm³/g (0.1-1.0 cm³/g, or 0.1-0.8 cm³/g, or 0.1-0.6 cm³/g, or 0.1-0.5 cm³/g, or 0.2-1.0 cm³/g, or 0.2-0.8 cm³/g, or 0.2-0.6 cm³/g, or 0.2-0.5 cm³/g).

Embodiment 11. The catalyst composition of any of embodiments 1-10, wherein the porous support has a bimodal pore size distribution, with a first mode having a peak in the range of 3-20 nm and a second peak in the range of 50-200 nm, as measured by mercury intrusion porosimetry according to ASTM D4284.

Embodiment 12. The catalyst composition of any of embodiments 1-11, wherein the porous support has at least 90% of its pore volume in pores in the range of 3-200 nm as measured by mercury intrusion porosimetry according to ASTM D4284.

Embodiment 13. The catalyst composition of any of embodiments 1-12, wherein the porous support is present in the catalyst in an amount of at least 98 wt %, e.g., at least 97 wt %, or at least 98 wt % or at least 99 wt %.

Embodiment 14. The catalyst composition of any of embodiments 1-13, wherein the palladium is present in an amount of at least 0.007 wt %, e.g., at least 0.01 wt % or at least 0.015 wt %.

Embodiment 15. The catalyst composition of any of embodiments 1-14, wherein the palladium is present in an amount in the range of 0.005 wt % to 0.2 wt %, e.g., 0.005-0.1 wt %, or 0.005-0.05 wt %, or 0.005-0.02 wt %.

Embodiment 16. The catalyst composition of any of embodiments 1-14, wherein the palladium is present in an amount in the range of 0.007 wt % to 0.2 wt %, e.g., 0.007-0.1 wt %, or 0.005-0.05 wt %, or 0.005-0.02 wt %.

Embodiment 17. The catalyst composition of any of embodiments 1-14, wherein the palladium is present in an amount in the range of 0.01 to 0.2 wt %, e.g., 0.01 to 0.1 wt %, or 0.01 to 0.05 wt %, or 0.01 to 0.02 wt %.

Embodiment 18. The catalyst composition of any of embodiments 1-14, wherein the palladium is present in an amount in the range of 0.015 to 0.2 wt %, e.g., 0.015 to 0.1 wt %, or 0.015 to 0.05 wt %, or 0.015 to 0.03 wt %.

Embodiment 19. The catalyst composition of any of embodiments 1-18, wherein the cesium is present in an amount of at least 0.02 wt %, e.g., at least 0.05 wt %, at least 0.07 wt %, or at least 0.1 wt %.

Embodiment 20. The catalyst composition of any of embodiments, 1-18, wherein the cesium is present in an amount in the range of 0.01 to 2 wt %, e.g., 0.01 to 1.5 wt %, or 0.01 to 1 wt %, or 0.01 to 0.5 wt %, or 0.01 to 0.3 wt %, or 0.01 to 0.1 wt %.

Embodiment 21. The catalyst composition of any of embodiments 1-18, wherein the cesium is present in an amount in the range of 0.02 to 2 wt %, e.g., 0.02 to 1.5 wt %, or 0.2 to 1 wt %, or 0.02 to 0.5 wt %, or 0.02 to 0.3 wt %, or 0.02 to 0.1 wt %.

Embodiment 22. The catalyst composition of any of embodiments 1-18, wherein the cesium is present in an amount in the range of 0.05 to 2 wt %, e.g., 0.05 to 1.5 wt %, or 0.05 to 1 wt %, or 0.05 to 0.5 wt %, or 0.05 to 0.3 wt %, or 0.05 to 0.15 wt %.

Embodiment 23. The catalyst composition of any of embodiments 1-18, wherein the cesium is present in an amount in the range of 0.07 to 2 wt %, e.g., 0.07 to 1.5 wt %, or 0.07 to 1 wt %, or 0.07 to 0.5 wt %, or 0.07 to 0.3 wt %, or 0.07 to 0.2 wt %.

Embodiment 24. The catalyst composition of any of embodiments 1-18, wherein the cesium is present in an amount in the range of 0.1 to 2 wt %, e.g., 0.1 to 1.5 wt %, or 0.1 to 1 wt %, or 0.1 to 0.5 wt %.

Embodiment 25. The catalyst composition of any of embodiments 1-24, wherein the amount of cesium is at least as much as the amount of palladium, e.g., least 1.5 times, or at least 2 times, or at least 2.5 times, or at least 3 times, or at least 3.5 times the amount of palladium.

Embodiment 26. The catalyst composition of any of embodiments 1-25, further comprising one or more metallic modifiers selected from silver, gold, zinc, tin, lead, cadmium, bismuth, gallium, and copper present in a total amount of up to 0.5 wt %.

Embodiment 27. The catalyst composition of any of embodiments 1-25, further comprising silver as a metallic modifier, present in a total amount of up to 0.5 wt %, e.g., up to 0.2 wt % or up to 0.1 wt %.

Embodiment 28. The catalyst composition of embodiment 26 or embodiment 27, wherein the metallic modifier is present in an amount in the range of 0.01 wt % to 0.5 wt % (e.g., in the range of 0.01 wt % to 0.2 wt %, or 0.01 to 0.1 wt %).

Embodiment 29. The catalyst composition of embodiment 26 or embodiment 27, wherein the metallic modifier is present in an amount in the range of 0.02 wt % to 0.5 wt % (e.g., in the range of 0.02 to 0.2 wt %, or 0.02 to 0.1 wt %).

Embodiment 30. The catalyst composition of any of embodiments 26-29, wherein the weight ratio of the metallic modifier to the palladium is at least 1:1 (e.g., at least 1.5:1, or 2:1).

Embodiment 31. The catalyst composition of any of embodiments 26-29, wherein the weight ratio of the metallic modifier to the palladium is at most about 12:1 (e.g. at most 10:1, or 5:1).

Embodiment 32. The catalyst composition of any of embodiments 26-29, wherein the weight ratio of the metallic modifier to the palladium is in the range of 1:1 to 12:1 (e.g., 1:1 to 10:1, or 1:1 to 5:1, or 1.5:1 to 12:1, or 1.5:1 to 10:1, or 1.5:1 to 5:1, or 2:1, to 12:1, or 2:1 to 10:1, or 2:1 to 5:1).

Embodiment 33. The catalyst composition of any of embodiments 1-32, wherein the palladium is localized at the outer surface of the catalyst.

Embodiment 34. The catalyst composition of any of embodiments 1-32, wherein at least 80 wt % (e.g., at least 90 wt %) of the palladium is within 1000 microns of an outer surface of the porous support.

Embodiment 35. The catalyst composition of any of embodiments 1-32, wherein at least 80 wt % (e.g., at least 90 wt %) of the palladium is within 800 microns of an outer surface of the porous support.

Embodiment 36. The catalyst composition of any of embodiments 1-32, wherein at least 80 wt % (e.g., at least 90 wt %) of the palladium is within 500 microns of an outer surface of the porous support.

Embodiment 37. The catalyst composition of any of embodiments 1-36, wherein the cesium is dispersed substantially throughout the support.

Embodiment 38. The catalyst composition of any of embodiments 1-36, wherein, if present, the metallic promoter is dispersed substantially throughout the support.

Embodiment 39. The catalyst composition of any of embodiments 1-38 comprising:
an alpha-alumina support, present in an amount of at least 98 wt %;
palladium, present in an amount in the range of 0.01 wt % to 0.1 wt % based on the weight of the catalyst composition;
cesium, present in an amount of at least 1.5 times that of palladium; and silver, present in an amount of at least 1.5 times that of palladium.

Embodiment 40. The catalyst composition of any of embodiments 1-38 comprising:
an alpha-alumina support, present in an amount of at least 98 wt %;
palladium, present in an amount in the range of 0.01 wt % to 0.1 wt % based on the weight of the catalyst composition;
cesium, present in an amount of at least 1.5 times that of palladium; and silver, present in an amount of at least 1.5 times that of palladium;
wherein the cesium and silver are substantially distributed throughout the support; and wherein at least 80 wt % (e.g., at least 90 wt %) of the palladium is within 1000 microns of an outer surface of the porous support.

Embodiment 41. The catalyst composition of embodiment 39 or embodiment 40, wherein palladium is present in an amount in the range of 0.01 wt % to 0.05 wt %.

Embodiment 42. The catalyst composition of any of embodiments 39-41, wherein at least 90 wt % of the palladium is within 800 microns of an outer surface of the porous support.

Embodiment 43. The catalyst composition of any of embodiments 39-42, wherein cesium is present in an amount in the range of 0.02 wt % to 0.5 wt % based on the weight of the catalyst. Embodiment 44. The catalyst composition of any of embodiments 39-43, wherein silver is present in an amount in the range of 0.02 wt % to 0.15 wt % based on the weight of the catalyst.

Embodiment 45. The catalyst composition of any of embodiments 1-44, wherein the catalyst composition has a BET surface area of no more than no more than 7 m²/g.

Embodiment 46. The catalyst composition of any of embodiments 1-44, wherein the catalyst composition has a BET surface area in the range of 1-10 m²/g (e.g., in the range of 1-7 m²/g, or 2-10 m²/g, or 2-7 m²/g, or 5-10 m²/g).

Embodiment 47. The catalyst composition of any of embodiments 1-46, wherein the catalyst composition has a pore volume of at least 0.1 cm³/g, e.g., at least 0.2 cm³/g.

Embodiment 48. The catalyst composition of any of embodiments 1-46, wherein the catalyst composition has a pore volume in the range of 0.1-1.0 cm³/g (e.g., 0.1-1.0 cm³/g, or 0.1-0.8 cm³/g, or 0.1-0.6 cm³/g, or 0.1-0.5 cm³/g, or 0.2-1.0 cm³/g, or 0.2-0.8 cm³/g, or 0.2-0.6 cm³/g, or 0.2-0.5 cm³/g).

Embodiment 49. The catalyst composition of any of embodiments 1-48, wherein the catalyst composition has a bimodal pore size distribution, with a first mode having a peak in the range of 3-20 nm and a second peak in the range of 50-200 nm, as measured by mercury intrusion porosimetry according to ASTM D4284.

Embodiment 50. The catalyst composition of any of embodiments 1-49, wherein the catalyst composition has at least 90% of its pore volume in pores in the range of 3-200 nm as measured by mercury intrusion porosimetry according to ASTM D4284.

Embodiment 51. The catalyst composition of any of embodiments 1-50, wherein no more than 25 mol %, e.g., no more than 10 mol % or no more than 5 mol %, of the palladium is present as Pd(0).

Embodiment 52. The catalyst composition of any of embodiments 1-51, wherein at least 75 mol %, for example, at least 90 mol % or at least 95 mol % of the palladium is in the form of one or more palladium oxides and/or hydroxides (e.g., including PdO).

Embodiment 53. The catalyst composition of any of embodiments 1-52, wherein no more than 25 mol %, e.g., no more than 10 mol % or no more than 5 mol %, of the silver is present as Ag(0).

Embodiment 54. The catalyst composition of any of embodiments 1-53, wherein at least 75 mol %, for example, at least 90 mol % or at least 95 mol % of the silver is in the form of one or more silver oxides and/or hydroxides (e.g., including Ag₂O and/or AgO).

Embodiment 55. The catalyst composition of any of embodiments 1-54, wherein no more than 25 mol %, e.g., no more than 10 mol % or no more than 5 mol %, of the cesium is present as Cs(0).

Embodiment 56. The catalyst composition of any of embodiments 1-55, wherein at least 75 mol %, for example, at least 90 mol % or at least 95 mol % of the cesium is in the form of one or more cesium oxides and/or hydroxides (e.g., including Cs₂O).

Embodiment 57. The catalyst composition of any of embodiments 1-56, in calcined form.

Embodiment 58. A method of making a catalyst composition (e.g., according to any of embodiments 1-57), the method comprising:
providing a porous support;
contacting the porous support with a first solution comprising a palladium compound and optionally a metallic compound;
drying and calcining the porous support to provide a palladium-impregnated support;
contacting the palladium impregnated support with a second solution comprising a cesium compound; and
drying and calcining the palladium-impregnated support to provide the catalyst composition.

Embodiment 59. The method of embodiment 58, wherein the palladium compound is a palladium salt (e.g., palladium nitrate, palladium phosphate, or palladium sulfate).

Embodiment 60. The method of embodiment 58 or embodiment 59, wherein, if present, the metallic compound is a metallic salt.

Embodiment 61. The method of any of embodiments 58-60, wherein, if present, the metallic compound is a metallic salt independently selected from a silver salt, a gold salt, a zinc salt, a tin salt, a lead salt, a cadmium salt, a bismuth salt, a gallium salt, and a copper salt.

Embodiment 62. The method of any of embodiments 58-61, wherein, if present, the metallic compound is a silver salt (e.g., silver nitrate, silver phosphate, or silver sulfate).

Embodiment 63. The method of any of embodiments 58-62, wherein the first solution further comprises an acid (e.g., nitric acid, phosphoric acid, or sulfuric acid).

Embodiment 64. The method of any of embodiments 58-63, wherein contacting the porous support with the first solution occurs for a time in the range of 30 minutes to 3 hours (e.g., in the range of 30 minutes to 2 hours, or 30 minutes to 1 hour, or 1 hour to 3 hours, or 1 hour to 2 hours).

Embodiment 65. The method of any of embodiments 58-64, wherein contacting the porous support with the first solution occurs at an ambient temperature.

Embodiment 66. The method of any of embodiments 58-65, wherein calcining the porous support occurs at a temperature in the range of 450° C. to 650° C. (e.g., in the range of 450 to 600° C., or 450 to 550° C., or 500 to 650° C., or 500 to 600° C., or 500 to 550° C.).

Embodiment 67. The method of any of embodiments 58-66, wherein calcining the porous support occurs for a time in the range of 1 hour to 4 hours (e.g., in the range of 1 to 3 hours, or 1 to 2 hours, or 2 to 4 hours, or 2 to 3 hours).

Embodiment 68. The method of any of embodiments 58-67, wherein the cesium compound is a cesium hydroxide.

Embodiment 69. The method of any of embodiments 58-68, wherein contacting the palladium-impregnated support with the second solution occurs for a time in the range of 30 minutes to 3 hours (e.g., in the range of 30 minutes to 2 hours, or 30 minutes to 1 hour, or 1 hour to 3 hours, or 1 hour to 2 hours).

Embodiment 70. The method of any of embodiments 58-69, wherein contacting the palladium-impregnated support with the second solution occurs at an ambient temperature.

Embodiment 71. The method of any of embodiments 58-70, wherein calcining the palladium-impregnated support occurs at a temperature in the range of 350° C. to 550° C. (e.g., in the range of 350 to 500° C., or 350 to 450° C., or 400 to 550° C., or 400 to 500° C.).

Embodiment 72. The method of any of embodiments 58-71, wherein calcining the palladium impregnated support occurs at a time in the range of 1 hour to 4 hours (e.g., in the range of 1 to 3 hours, or 1 to 2 hours, or 2 to 4 hours, or 2 to 3 hours).

Embodiment 73. A catalyst composition (e.g., according to any of embodiments 1-57) made by the method of any of embodiments 58-72.

Embodiment 74. A method for selective hydrogenation of alkyne (e.g., acetylene and/or methylacetylene) in an olefin feed stream comprising hydrogen and alkyne, the method comprising contacting the olefin feed stream with a catalyst composition according to any of embodiments 1-57 and 73.

Embodiment 75. The method of embodiment 74, wherein the olefin feed stream comprises C1-C3 hydrocarbons.

Embodiment 76. The method of embodiment 74, wherein the olefin feed stream comprises ethylene and acetylene.

Embodiment 77. The method of embodiment 74, wherein the olefin feed stream comprises propylene and methylacetylene.

Embodiment 78. The method of any of embodiments 74-77, wherein the olefin feed stream comprises at least 10 mol % olefin, e.g., at least 20 mol % olefin (e.g., wherein the olefin is ethylene and/or propylene).

Embodiment 79. The method of embodiment 78, wherein there is no more than 70 mol % olefin, e.g., no more than 60 mol % olefin, or no more than 50 mol % olefin in the olefin feed stream.

Embodiment 80. The method of any of embodiments 74-79, wherein the olefin feed stream comprises at least 1 ppm alkyne, e.g., at 10 ppm alkyne, or at least 100 ppm alkyne, or at least 500 ppm alkyne (e.g., wherein the alkyne is acetylene and/or methylacetylene).

Embodiment 81. The method of embodiment 80, wherein there is no more than 2 mol % alkyne, e.g., no more than 1.5 mol % alkyne, or no more than 1 mol % alkyne in the olefin feed stream.

Embodiment 82. The method of any of embodiments 74-81, wherein the olefin feed stream comprises at least 5 mol % hydrogen, e.g., at least 10 mol % hydrogen.

Embodiment 83. The method of embodiment 82, wherein the olefin feed stream comprises no more than 40% hydrogen, e.g., no more than 30% hydrogen, or no more than 20% hydrogen.

Embodiment 84. The method of any of embodiments 74-83, wherein the olefin feed stream includes one or more inerts (e.g., methane, ethane, propane, desirably methane), present in an amount of at least 5 mol %, e.g., at least 10 mol % or at least 20 mol %.

Embodiment 85. The method of embodiment 84, wherein no more than 70 mol % of one or more inerts (e.g., methane, ethane, propane, desirably methane), e.g., no more than 60 mol % or no more than 50 mol %, is present in the olefin feed stream.

Embodiment 86. The method of any of embodiments 74-85, wherein the olefin feed stream is a front end olefin feed stream, and the hydrogenation is a front end hydrogenation.

Embodiment 87. The method of any of embodiments 74-77, wherein the olefin feed stream comprises hydrogen in an amount of at least 0.1 mol %, e.g., at least 0.2 mol %, or at least 0.5 mol %, or at least 1 mol %.

Embodiment 88. The method of embodiment 87, wherein the olefin feed stream comprises hydrogen in an amount of no more than 20 mol %, e.g., no more than 15 mol %, or no more than 7 mol %, or no more than 5 mol %.

Embodiment 89. The method of any of embodiments 74-77, 87 and 88, wherein the olefin feed stream includes at least 1 ppm alkyne, e.g., at least 10 ppm alkyne, or at least 100 ppm alkyne, or at least 500 ppm alkyne (e.g., wherein the alkyne is acetylene and/or methylacetylene).

Embodiment 90. The method of embodiment 89, wherein there is no more than 2 mol % alkyne, e.g., no more than 1.5 mol % alkyne, or no more than 1 mol % alkyne, in the olefin feed stream.

Embodiment 91. The method of any of embodiments 71-77 and 87-90, wherein the olefin feed stream comprises at least 20 mol % olefin, e.g., at least 50 mol % olefin, or at least 70 mol % olefin (e.g., wherein the olefin is ethylene and/or propylene).

Embodiment 92. The method of embodiment 91, wherein there is no more than 90 mol % olefin, e.g., no more than 80 mol % olefin, or no more than 70 mol % olefin, in the olefin feed stream.

Embodiment 93. The method of any of embodiments 74-77 and 87-92, wherein one or more inerts (e.g., methane, ethane, propane, desirably methane) are present in an amount of at least up to 30 mol %, e.g., up to 20 mol % or up to 10 mol %.

Embodiment 94. The method of any of embodiments 74-77 and 87-93, wherein the olefin feed stream is a back-end or tail-end olefin feed stream, and the hydrogenation is a back-end or tail-end hydrogenation.

Embodiment 95. The method of any of embodiments 74-94, wherein the molar ratio of hydrogen to alkyne is in the range of from about 1 to about 1000 (e.g., in the range of from about 1.1 to about 800).

Embodiment 96. The method of any of embodiments 74-95, wherein the olefin feed stream further comprises carbon monoxide.

Embodiment 97. The method of any of embodiments 74-96, wherein the contacting is performed at a temperature is in the range of 20 to 150° C. (e.g., in the range of 40 to 150° C., or 60-150° C., or 20 to 130° C., or 40 to 130° C., or 60-130° C., or 20-110° C., or 40 to 110° C., or 60-110° C.).

Embodiment 98. The method of any of embodiments 74-97, wherein the contacting is performed at a GSHV in the range of 5,000 to 20,000 h⁻¹ (e.g., in the range of 5000 h⁻¹ to 15,000 h⁻¹, or 8,000 to 20,000 h⁻¹, or 8000-15,000 h⁻¹).

Embodiment 99. The method of any of embodiments 74-98 wherein the contacting is performed at a pressure in the range of 100 to 500 psig (e.g., in the range of 100 to 400 psig, or 200 to 500 psig, or 200 to 400 psig).

Embodiment 100. The method of any of embodiments 74-99, wherein the method of selective hydrogenation of alkyne provides an output olefin stream having less than 25 ppm alkyne.

Embodiment 101. The method of any of embodiments 74-100, conducted such that the amount of alkyne in the output olefin stream is no more than 2% of the amount of the corresponding alkene of the olefin feed stream.

Embodiment 102. The method of any of embodiments 74-101, wherein the method of selective hydrogenation of alkyne has an operating window of at least 10 degrees (e.g., of at least 15 degrees, or at least 20 degrees, or at least 25 degrees, or at least 30 degrees, or at least 35 degrees).

Embodiment 103. The method of any of embodiments 74-102, wherein the contacting of the alkyne and hydrogen with the catalyst composition occurs in a reactor, and the method further comprises loading the catalyst composition into the reactor.

Embodiment 104. The method of embodiment 103, wherein when the catalyst composition is loaded into the reactor, no more than 25 mol %, e.g., no more than 10 mol % or no more than 5 mol %, of the palladium is present as Pd(0).

Embodiment 105. The method of embodiment 103 or embodiment 104, wherein when the catalyst composition is loaded into the reactor, at least 75 mol %, for example, at least 90 mol % or at least 95 mol % of the palladium is in the form of one or more palladium oxides and/or hydroxides (e.g., including PdO).

Embodiment 106. The method of any of embodiments 103-105, wherein when the catalyst composition is loaded into the reactor, no more than 25 mol %, e.g., no more than 10 mol % or no more than 5 mol %, of the silver is present as Ag(0).

Embodiment 107. The method of any of embodiments 103-106, wherein when the catalyst composition is loaded into the reactor, at least 75 mol %, for example, at least 90 mol % or at least 95 mol % of the silver is in the form of one or more silver oxides and/or hydroxides (e.g., including Ag₂O and/or AgO).

Embodiment 108. The method of any of embodiments 103-107, wherein when the catalyst composition is loaded into the reactor, no more than 25 mol %, e.g., no more than 10 mol % or no more than 5 mol %, of the cesium is present as Cs(0).

Embodiment 109. The method of any of embodiments 103-108, wherein when the catalyst composition is loaded into the reactor, at least 75 mol %, for example, at least 90 mol % or at least 95 mol % of the cesium is in the form of one or more cesium oxides and/or hydroxides (e.g., including Cs₂O).

Embodiment 110. The method of any of embodiments 103-109, wherein when the catalyst composition is loaded into the reactor it is in a calcined form.

Embodiment 111. The method of any of embodiments 74-110, further comprising, before contacting the olefin stream with the catalyst composition, activating the catalyst composition by reducing (e.g., with hydrogen).

What is claimed is:

1. A catalyst composition comprising:
a porous support; and, disposed on the support:
palladium, present in an amount of at least 0.005 wt %, e.g., in the range of 0.005 wt % to 0.2 wt %, based on the weight of the catalyst composition; and
cesium, present in an amount of at least 0.01 wt %, e.g., in the range of 0.01 wt % to 2 wt % based on the weight of the catalyst composition.

2. The catalyst composition of claim 1, wherein the porous support is an alpha-alumina support, e.g., comprising at least 95 wt % alpha-alumina.

3. The catalyst composition of claim 1, wherein the porous support has a BET surface area in the range of 1-10 m²/g.

4. The catalyst composition of claim 1, wherein the palladium is present in an amount in the range of 0.005 wt % to 0.2 wt %.

5. The catalyst composition of claim 1, wherein the cesium is present in an amount in the range of 0.05 to 0.6 wt %.

6. The catalyst composition of claim 1, wherein the cesium is present in an amount in the range of 0.1 to 2 wt %.

7. The catalyst composition of claim 1, wherein the amount of cesium is at least as much as the amount of palladium.

8. The catalyst composition of claim 1, further comprising silver as a metallic modifier, present in a total amount of up to 0.5 wt %.

9. The catalyst composition of claim 9, wherein the metallic modifier is present in an amount in the range of 0.02 wt % to 0.5 wt %.

10. The catalyst composition of claim 10, wherein at least 80 wt % of the palladium is within 1000 microns of an outer surface of the porous support.

11. The catalyst composition of claim 1, wherein at least 80 wt % (e.g., at least 90 wt %) of the palladium is within 1000 microns of an outer surface of the porous support and the cesium is dispersed substantially throughout the support.

12. The catalyst composition of claim 1, comprising:
an alpha-alumina support, present in an amount of at least 98 wt %;
palladium, present in an amount in the range of 0.01 wt % to 0.1 wt % based on the weight of the catalyst composition;
cesium, present in an amount of at least 1.5 times that of palladium; and
silver, present in an amount of at least 1.5 times that of palladium.

13. The catalyst composition of claim 1 comprising:
an alpha-alumina support, present in an amount of at least 98 wt %;
palladium, present in an amount in the range of 0.01 wt % to 0.1 wt % based on the weight of the catalyst composition;
cesium, present in an amount of at least 1.5 times that of palladium; and
silver, present in an amount of at least 1.5 times that of palladium;
wherein the cesium and silver are substantially distributed throughout the support; and
wherein at least 80 wt % of the palladium is within 1000 microns of an outer surface of the porous support.

14. The catalyst composition of claim 1, wherein the catalyst composition has a BET surface area in the range of 1-10 m²/g.

15. The catalyst composition of claim 1, wherein no more than 5 mol % of the palladium is present as Pd(0).

16. The catalyst composition of claim 1, wherein at least 90 mol % of the palladium is in the form of one or more palladium oxides and/or hydroxides.

17. The catalyst composition of claim 1, in calcined form.

18. A method of making a catalyst composition according to claim 1, the method comprising:
providing a porous support;
contacting the porous support with a first solution comprising a palladium compound and optionally a metallic compound;
drying and calcining the porous support to provide a palladium-impregnated support;
contacting the palladium impregnated support with a second solution comprising a cesium compound; and
drying and calcining the palladium-impregnated support to provide the catalyst composition.

19. A method for selective hydrogenation of alkyne in an olefin feed stream comprising alkyne and hydrogen, the method comprising contacting the olefin feed stream with a catalyst composition according to claim 1.

20. The method of claim 19, wherein the olefin feed stream comprises 10-70 mol % olefin, 1 ppm-2 mol % alkyne, and 5-40% hydrogen.

21. The method of claim 19, wherein the olefin feed stream is a front end olefin feed stream, and the hydrogenation is a front end hydrogenation.

22. The method of claim 19, wherein the olefin feed stream comprises at least 50 mol % olefin, 1-20 mol % hydrogen and 1 ppm-2 mol % alkyne.

23. The method of claim 19, wherein the olefin feed stream is a back-end or tail-end olefin feed stream, and the hydrogenation is a back-end or tail-end hydrogenation.

24. The method of claim 19, wherein the contacting of the alkyne and hydrogen with the catalyst composition occurs in a reactor, and the method further comprises loading the catalyst composition into the reactor.

25. The method of claim 24, wherein when the catalyst composition is loaded into the reactor, no more than 5 mol % of the palladium is present as Pd(0).

26. The method of claim 24, wherein when the catalyst composition is loaded into the reactor, at least 90 mol % of the palladium is in the form of one or more palladium oxides and/or hydroxides (e.g., including PdO).

27. The method of claim 24, wherein when the catalyst composition is loaded into the reactor it is in a calcined form.
